(19) United States
(12) Patent Application Publication
(10) Pub. No.: US 2012/0060767 A1
(43) Pub. Date: Mar. 15, 2012

(54) Progesterone receptor membrane component 1 (PGRMC1) as an indicator of fertility in animals

(75) Inventor: John J. Peluso, Avon, CT (US)

(73) Assignee: University of Connecticut, Farmington, CT (US)

(21) Appl. No.: 13/135,851

(22) Filed: Jul. 15, 2011

Related U.S. Application Data
(60) Provisional application No. 61/364,711, filed on Jul. 15, 2010.

(51) Int. Cl. A01K 67/00; C12Q 1/68; G01N 33/566

(52) U.S. Cl. 119/712; 436/501; 435/7.21; 435/6.12

(57) ABSTRACT

Methods of evaluating fertility of an animal based on determination of a PGRMC1 characteristic that correlates with animal fertility. In one embodiment, a sample is obtained from an animal, a PGRMC1 characteristic is determined and compared to a baseline PGRMC1 characteristic, wherein a variation between the determined PGRMC1 characteristic and the baseline characteristic indicates a level of fertility of the animal. A PGRMC1 characteristic can be one or more of PGRMC1 expression, transcription, translation, amino acid sequence, nucleic acid sequence, post-translational modification, cell localization or tissue localization. A sample can be a cell sample, a tissue sample, a blood sample, a lymphocyte sample, an oocyte sample, or a sperm sample. The animal can be male or female. In one embodiment, a variation of PGRMC1 nucleic acid sequence indicates reduced fertility of the animal.

### RELATED APPLICATIONS

The present application claims the benefit of U.S. Provisional Patent Application Ser. No. 61/364,711, filed Jul. 15, 2010, which is herein incorporated by reference in its entirety.

### STATEMENT OF GOVERNMENT SUPPORT

This invention was made with government funding under the NIH grant entitled “PAIRBP1 & PGRMC1 act as a membrane receptor complex to mediate P4's ovarian action” (NIH grant number R01 HD 052740). The government has certain rights in the invention.

### FIELD

Embodiments of the present invention relate to methods of evaluating, assessing or predicting animal fertility based on analysis of progesterone receptor membrane component 1 (PGRMC1).

### BACKGROUND

Methods for assessing animal fertility are useful in a variety of fields, such as animal husbandry, animal science, zoology, veterinary science, medicine, biological and medical research. For example, an ability to assess animal fertility in order to select animals with higher fertility potential is useful for improving the efficiency of breeding of domestic animals, experimental animals, captive wild animals or any animals of economic or scientific interest.

Dairy farming is one example of a field where a method for assessing animal fertility is useful. Dairy farmers around the world are under tremendous economic pressure to produce milk products at the lowest possible price. This economic pressure results in extremely small profit margins that are reaching a point that force many small farms out of the dairy business. The profitability of a dairy farm in many ways is reflected by pregnancy rates (PR) of dairy cattle. For example, a farm with a PR of 17% has a profit of $880 per cow. The profit decreases to $630 per cow if the PR is 12%. Thus, for a typical dairy farm with 200 head of cattle, increasing the PR from 12 to 17% would increase the profit margin by $50,000 per year, or by 40%.

Typically, in dairy farming, a heifer is bred after two years, and it is desirable for it to conceive and begin to produce milk. In high fertility cows, this cycle of pregnancy and lactation (i.e. milk production) typically continues for ten to twelve years. Unfortunately, there is a population of cows that have extended intervals between pregnancies, lowering the PR and depriving the farmer of both a valuable calf and milk production. These cows are generally culled when they are between four to six years of age.

At present, few dairy herds have a PR of a 17%. There are numerous factors that contribute to PR. One important factor in the PR calculation is fertility, which is defined in some instances as the ability to ovulate mature, fertilizable oocytes that ultimately develop into healthy calves. PR could be dramatically improved by a method capable of identifying calves with high fertility potential prior to puberty. This would allow for those calves or heifers with low fertility potential to be culled, thereby eliminating the expense associated with raising them to maturity. However, conventional methods do not allow for accurate prediction of fertility in cattle prior to puberty.

To date there are not ways to predict fertility other than directly monitoring fertility of mature cattle. No genetic tests are available at present to assess animal fertility.

What is needed is a test to evaluate the probability of whether an individual immature female animal will produce viable oocytes capable of being fertilized and developing into viable embryos (“functional oocytes”) once she obtains puberty. What is also needed is a test to evaluate a probability of whether an individual male's ability to produce sperm cells capable of fertilizing oocytes so that they can develop into viable embryos (“functional sperm cells”). What is also needed is a test that would predict the probability of a successful reproductive outcome for an individual animal or a breeding couple, based on the ability to produce functional oocytes, functional sperm cells, or both. What is also needed is a test that would allow customization of breeding approaches, assisted reproductive techniques or procedures based on an animal's ability to produce functional oocytes, functional sperm, or both. A test is also needed that would be relatively non-invasive, relatively inexpensive and easy to use, or using samples that are relatively easy to obtain, such as blood samples, so that the test could be administered or the samples obtained safely and with minimal training and effort by animal handlers or other personnel.

### SUMMARY

Disclosed herein are embodiments of methods of assessing and/or evaluating fertility of animals. For example, disclosed herein are methods of assessing or evaluating the ability of animals to produce functional gametes (eggs or sperm) by analyzing characteristics of progesterone receptor membrane component-1 (PGRMC1). Some examples of PGRMC1 characteristics used in embodiments of the methods disclosed herein are levels of PGRMC1 expression, including transcription and translation, protein or nucleic acid structure or sequence, post-translational modifications, localization in cells and tissues. In some embodiments, methods disclosed herein evaluate, measure, assess or determine variation of PGRMC1 characteristics. In the context of the methods disclosed herein, PGRMC1 variation encompasses, without limitation, variation in PGRMC1 expression, including transcription and translation, variation of protein or nucleic acid structure, variation of post-translational modifications, or variation in localization in cells and tissues. Generally, variation is understood to imply the differences in certain PGRMC1 characteristics, such as those listed above, identified in a particular individual with respect to the baseline, typical, or average characteristics, or temporal variation in PGRMC1 characteristics in an individual animal, or in a cell or tissue, including a cell or a tissue sample obtained from an animal.

In one embodiment, analysis of PGRMC1 characteristics and/or their variation is used to evaluate or predict the capacity of a female to produce functional oocytes, or oocytes that are able to be fertilized and ultimately develop into a healthy offspring. In another embodiment, analysis of PGRMC1 characteristics and/or their variation is used to predict the capacity of a male to produce functional sperm cells, or sperm cells that are able to fertilize oocytes, which are then able to develop into healthy offspring. In yet another embodiment, analysis of PGRMC1 characteristics and/or their variation is used to predict outcomes and/or improve the efficiency of assisted reproduction techniques or procedures, such as artificial insemination or in vitro fertilization protocols. In one more embodiments, a method of selecting functional gametes is provided by analyzing PGRMC1 characteristics and/or their variation.

Also provided are methods of assessing, evaluating or monitoring of fertility of individual animals or groups of animals (such as herds) by analyzing PGRMC1 characteristics and/or their variation. For example, one embodiment provided herein relates to a method of predicting fertility of domestic animals, such as, but not limited to, dairy cattle. In some embodiments, the above assessing, evaluating or monitoring of fertility of individual animals or of a group of animals is used for improving the efficiency or predicting outcomes of animal breeding. In another embodiment, the above assessing, evaluating or of fertility is used for selecting animals with desired fertility characteristics. Such methods are useful, for example, for in dairy cattle management for improving the pregnancy rate (PR) of a herd.

In other embodiments, the above assessing of fertility of individual animals or of a group of animals is used for diagnosis or treatment of infertility of an animal, for example, in the veterinary field. In one exemplary embodiment, a method of treating infertility is provided by improving the effectiveness of an infertility treatment or an assisted reproduction technique in an animal that includes analyzing PGRMC1 characteristics and/or their variation in the animal and modifying the treatment or the technique in accordance with the results of PGRMC1 analysis. These and other features and advantages of the present methods will become apparent after a review of the following detailed description of the disclosed embodiments and the appended claims.

### BRIEF DESCRIPTION OF THE FIGURES

FIG. 1 is a reproduction of an image of a Western blot showing PGRMC1 expression in bovine ovarian cortex and bovine oocytes. A Western blot analysis conducted in the absence of the PGRMC1 antibody, is shown as a negative control (−).

FIG. 2 is a reproduction of epi-fluorescent images of cells showing localization of PGRMC1 in bovine and mouse germinal vesicle-stage oocytes. PGRMC1 is shown in red. PGRMC1 was detected throughout the oocyte, but is highly concentrated within the germinal vesicle (arrows) of both bovine and mouse oocytes. Negative controls were conducted on both bovine and mouse oocytes by omitting the primary antibody, which did not reveal any staining. Mag bar is 20μ.

FIG. 3 is a reproduction of confocal images of cells showing changes in the localization of PGRMC1 during bovine oocyte maturation. A negative control, which was conducted in the absence of the PGRMC1 antibody, is shown in the upper left panel. The DNA was stained with DAPI and is shown in blue, and PGRMC1 is shown in red. Oocytes were collected at the germinal vesicle stage (GV), after the breakdown of the GV (i.e. GVBD), prometaphase 1 (Pro-MI), metaphase I (Meta I), anaphase I (Ana I), telophase I (Telo I) and metaphase II (Meta II). The images of metaphase I, anaphase I and metaphase II are shown in a polar view (i.e. looking down onto the metaphase chromosomes). The images of the anaphase I and telophase I oocytes are in the lateral view (i.e. looking at the side of the anaphase I and telophase I chromosomes).

FIG. 4 is a reproduction of confocal images of cells showing colocalization of PGRMC1 and Aurora B on metaphase II chromosomes in bovine oocytes. The chromosomes are shown in blue, PGRMC1 in red and Aurora B in green. A merged image of PGRMC1 and Aurora B staining is shown in the lower right panel. The areas that appear orange-yellow are areas where PGRMC1 and Aurora B colocalize. Mag bar=10μ.

FIG. 5 is a reproduction of epi-fluorescent images of cells showing PGRMC1 localization in a bovine zygote. PGRMC1 (red) is only detected within nucleolar-like structures of the female and male pronuclei.

FIG. 6 is a reproduction of epi-fluorescent images of the cells showing localization of PGRMC1 in a bovine blastocyst. PGRMC1 is shown in red (A) and nuclei are shown in blue (B). A merged image is shown in C.

FIG. 7 is a bar graph representing the data on an effect of PGRMC1 antibody injection on bovine oocyte maturation. On the x-axis, the following abbreviations are used: GVBD: germinal vesicle breakdown: proMI: prometaphase I; MI: metaphase I; A/T: anaphase/telophase; MII: metaphase II. * indicates a difference between control and PGRMC1 antibody injection (p<0.05).

FIG. 8 is a reproduction of epi-fluorescent images of cells showing the effect of PGRMC1 antibody injection on the alignment of chromosomes along the metaphase I during bovine oocyte maturation in vitro. Oocytes were stained with propididum iodide (red) to reveal the chromosomes. Panel A shows chromosomes precisely arranged in a metaphase I plate, which is typical of IgG injected oocytes. Panel B shows a bovine oocyte 24 h after being injected with PGRMC1 antibody, with the chromosomes not aligned and the metaphase plate disorganized.

FIG. 9 is a reproduction of a fluorescent image of granulosa/luteal cells in culture showing protein expression as a result an infection with adenovirus-PGRMC1-GFP construct a MOI of 1×10⁻⁷, resulting in nearly 100% of the cells expressing PGRMC1-GFP fusion.

FIG. 10 is a reproduction of a gel showing detection of bovine PGRMC1 in mRNA prepared from bovine lymphocytes (white blood cells; WB).

### DETAILED DESCRIPTION

Disclosed herein are embodiments of methods of assessing or evaluating fertility, including the ability to produce functional gametes, of animals that include analyzing characteristics of PGRMC1. In some embodiments, methods of assessing or evaluating fertility include determination and analysis of variation of one or more PGRMC1 characteristic. It is understood that analyzing PGRMC1 characteristics and/or their variation allows assessment of an oocyte's capacity to undergo maturation, fertilization and normal embryological development, and of a sperm cell's capacity to fertilize an oocyte, which is then able to undergo normal embryological development. Gametes having such capacities are referred as “functional gametes.” It is understood that the ability of an animal to produce functional gametes correlates with fertility, including, but not limited, the probability of conceive and carry a pregnancy to term, and with the success rate of assisted reproductive techniques and fertility treatments, including the outcome of artificial insemination or in vitro fertilization.

It is discovered and described herein that PGRMC1 plays an essential role in regulating oocyte development and function. In particular, it is discovered that PGRMC1 influences an oocyte's capacity to undergo maturation, fertilization and normal embryological development. In mammalian ovaries, oocytes are arrested in prophase of the first meiotic division until they reach their full size and enter the preovulatory meiotic maturation process. Nuclear meiotic maturation of oocytes includes condensation of chromosomes, germinal vesicle breakdown (GVBD), progression through prometaphase I and metaphase I, the transition throughout anaphase I and telophase I and an arrest at metaphase II. This process is coordinated by several kinases and initiates when the luteinizing hormone surge stimulates the resumption of meiosis in one or more oocytes depending on the species. In mammalian oocytes, fully-grown oocytes are capable of resuming meiosis in vitro, and then of being fertilized and developing to the blastocyst stage. During oocyte meiotic division, the meiotic spindle asymmetrically segregates homologous pairs into the secondary oocyte and the polar body, then the egg arrests to metaphase II until fertilization occurs.

Defects in spindle formation can generate chromosome instability and aneuploidy, a condition known to be the major cause of miscarriages and birth defects. For example, aneuploidy is observed in a large percentage (as high as 40%) of embryos derived from patients undergoing infertility treatment. To guarantee the correct function of the spindle, the activity and localization of spindle-associated proteins has to be strictly regulated in time and space. The studies described herein are the first to show that PGRMC1 is 1) expressed in mammalian oocytes and 2) associated with the meiotic spindle. Moreover, it is discovered and described herein that PGRMC1's localization dramatically changes during oocyte maturation, fertilization and early preimplantation development.

For example, PGRMC1 localizes to the centromere of chromosomes in metaphase I and metaphase II oocytes. Localization to centromeres is significant because centromeres are involved in the attachment of microtubules, which function to separate the chromosomes during the metaphase to telophase transition. The temporal changes in the expression of PGRMC1 during oocyte maturation, fertilization and early preimplantation development indicate an important role for PGRMC1. For example, dramatic changes in localization indicate that PGRMC1 is involved in regulating the separation of chromosomes during oocyte maturation. PGRMC1 is also found in sperm cells, as discussed, for example, in Lösel et al., “Classic and Non-classic Progesterone Receptors are Both Expressed in Human Spermatozoa,” Horm. Metab. Res. 37:10-14, 2005; Lösel et al. “Porcine Spermatozoa Contain More than One Membrane Progesterone Receptor,” Int Journ. Of Biochemistry and Cell Biology, 36:1532-1541, 2004. Accordingly, PGRMC1 protein and nucleic acids encoding PGRMC1 are useful as markers of a capacity of an animal to correctly undergo various processes involved in production and functioning of gametes, including the processes discussed in above, as well as of the functioning of zygotes and embryonic development.

Embodiments of methods of analyzing or testing of PGRMC1 characteristics and/or their variation are described herein. Embodiments of the methods described herein are useful for assessing functioning of the biological processes discussed above, some or all of which are understood to be important for animal fertility. In other words, certain embodiments of the methods described herein allow assessing the capacity of oocytes and sperm to undergo various processes affecting fertility, such as, but not limited to, meiosis, fertilization and cell differentiation. Thus, certain embodiments of the described methods allow for assessment of the ability of the oocytes and sperm to undergo fertilization, implantation, as well as to generate normal embryos.

### PGRMC1 Sequences and Mutations

Polypeptide and nucleic acid sequences for PGRMC1 are known in the art and can be obtained from publicly available sources. For example, such as, polypeptide and nucleic acid sequence databases are available through the National Center for Biotechnology Information (NCBI).

An example of a polypeptide and a transcript nucleotide sequence for bovine PGRMC1 is available as BT27888 from Bovine Genome Database at:

(SEQ ID NO: 1)
MAAEDVAATGADTSELESGGLLQEIFTSPLNLLLLGLCIFLLYKIVRGDQ
PAASDSDDDEPPPLPRLKRRDFTPAELRRFDGVQDPRILMAINGKVFDVT
KGRKFYGPEGPYGVFAGRDASRGLATFCLDKEALKDEYDDLSDLTPAQQE
TLSDWDSQFTFKYHHVGKLLKDGEEPTVYSDEEEPKDESTRKND
(SEQ ID NO: 2)
ATGGCTGCCGAGGATGTGGCGGCGACCGGCGCAGACACGAGCGAGCTCGA
GAGCGGCGGGCTGCTGCAAGAGATTTTCACGTCGCCGCTCAACCTGCTGC
TCCTTGGCCTTTGCATCTTTCTGCTCTACAAGATCGTGCGCGGGGACCAG
CCGGCGGCCAGCGATAGCGACGACGACGAGCCGCCCCCGCTGCCCCGCCT
TAAGCGGCGCGACTTCACCCCTGCCGAGCTGCGGCGCTTCGACGGCGTAC
AGGACCCGCGTATACTCATGGCCATCAATGGCAAGGTGTTCGACGTAACC
AAAGGCCGCAAGTTCTACGGGCCTGAGGGGCCGTATGGAGTCTTTGCTGG
AAGAGATGCATCCAGAGGCCTTGCCACCTTTTGCCTGGATAAGGAAGCGC
TGAAGGATGAGTACGATGACCTTTCCGACCTCACTCCTGCCCAGCAGGAG
ACCCTGAGTGACTGGGACTCTCAGTTCACTTTCAAGTACCATCATGTGGG
CAAACTGCTGAAGGACGGGGAGGAGCCCACCGTGTACTCAGACGAGGAGG
AGCCAAAAGATGAGAGCACTCGGAAGAATGAT

Naturally occurring mutations in PGRMC1 are known to exist. Such mutations have been identified and described in human females with known fertility problems. For example, the studies on alteration in expression, structure and function of PGRMC1 in females with premature ovarian failure are summarized in Mansouri, M. R., et al., “Alterations in the expression, structure and function of progesterone receptor membrane component-1 (PGRMC1) in premature ovarian failure,” Hum. Mol. Genet, 17(23):3776-83, 1998. Premature ovarian failure (POF) is a condition characterized by hypergonadotropic hypogonadism and amenorrhea in human females before the age of 40. In a scientific study, a mother and daughter with POF were identified both of whom carried an X; autosome translocation [t(X; 11)(q24; q13)] and had reduced expression levels of PGRMC1, as determined based on RNA transcript and protein levels. A human female with POF was also identified carrying a missense mutation (H165R) located in the cytochrome b5 domain of PGRMC1, which was shown to be associated with abolition of the binding of PGRMC1 to cytochrome P450 7A1 (CYP7A1). The H165R mutation is also known to attenuate PGRMC1's ability to mediate the anti-apoptotic action of progesterone in ovarian cells.

In addition to H165R, another missense mutation at amino acid 120 has been detected, which results in a complete loss of human PGRMC1's actions, as discussed in Peluso et al., “Progesterone receptor membrane component-1 (PGRMC1) is the mediator of progesterone's anti-apoptotic action in spontaneously immortalized granulosa cells as revealed by PGRMC1 small interfering ribonucleic acid treatment and functional analysis of PGRMC1 mutations,” Endocrinology 149:534-543, 2008 (“Peluso I”).

According to some embodiments of the methods described herein, at least some genetic alterations in PGRMC1 adversely affect the ability of the oocyte to mature, fertilize and undergo early preimplantation development. Accordingly, certain embodiments of the present method provide methods of assessing fertility that include testing of PGRMC1 in order to determine the presence of mutations in the PGRMC1-encoding nucleic acid sequences.

Some embodiments of the methods described herein allow for identification of PGRMC1 variability affecting animal fertility by analyzing PGRMC1 in animals of known lowered fertility. It is understood that by evaluating and/or determining PGRMC1 characteristics, such as expression level and genetic structure of PGRMC1, provides information that correlates with the potential fertility of an animal. For example, analysis of PGRMC1 sequences in animals of known lowered fertility, such as cows that have been prematurely culled from the herd based on their lowered fertility, allows for the identification of PGRMC1 mutations and/or variants that correlate with lowered fertility in animals. Thus identified mutations and/or variants serve as a basis of a genetic test for predicting the fertility of animals. In dairy farming, the genetic test allows, for example, to identify the calves with lowered fertility at an earlier stage than currently possible. This allows a farmer to conserve the resources by choosing to cull calves with lower fertility at an early stage and to improve the PR of the heard, thereby improving the profitability.

### PGRMC1 Characteristics

Embodiments of the methods provided herein evaluate one or more of the following PGRMC1 characteristics: structure and function of cells or tissues containing PGRMC1 protein and PGRMC1-encoding nucleic acids; expression of PGRMC1 protein; levels of PGRMC1 protein; location of expression of PGRMC1 protein; transcription of PGRMC1 mRNA; levels of PGRMC1 mRNA; location of PGRMC1 mRNA; structure and function of PGRMC1 protein; structure and function of PGRMC1-encoding nucleic acids, including DNA and RNA; interactions of PGRMC1 protein and PGRMC1-encoding nucleic acids in with other proteins, nucleic acids or other molecules; structure and function of chromatin and chromosomes containing PGRMC1-encoding nucleic acids.

### PGRMC1 Analysis or Testing

Evaluation of the foregoing characteristics according to embodiments of the methods disclosed herein can be performed in whole organisms, tissues, cells, samples obtained from organisms, tissues or cells, in various in situ, in vivo and in vitro systems, and in computational modeling systems (also referred to as in silico). All of the foregoing is collectively referred to as “PGRMC1 analysis” or “PGRMC1 testing.” Embodiments of the present methods utilize PGRMC1 testing in order to determine if variation in any of the foregoing PGRMC1 characteristics exists in an individual animal or a group of animals, or in a cell or a tissue; including a cell or a tissue sample obtained from an animal, as compared to average or normal characteristics existing in an animal population. Other embodiments of the present methods utilize PGRMC1 testing in order to determine temporal or spatial variation of PGRMC1 in an individual animal or a group of animals, or in a cell or a tissue, including a cell or a tissue sample obtained from an animal.

In certain embodiments of the methods described herein, analysis or testing of PGRMC1 characteristics and/or their variation is conducted in a sample obtained from an animal. A sample is a cell or tissue sample containing PGRMC1, PGRMC1-encoding nucleic acids, or both. One advantage of certain embodiments of the methods discussed herein is that PGRMC1 protein is generally expressed in oocytes and sperm, which permits assessment of fertility of both males and females. Another advantage of certain embodiments of the methods discussed herein is that PGRMC1 is present within blood cells, for example, lymphocytes, which allows for convenient and relatively non-invasive testing of a blood sample. However, testing of other cells and tissues where PGRMC1 is present, such as the sperm cells and the oocytes, can also be conducted. Testing of PGRMC1-encoding nucleic sequences can be conducted on any cells and tissues where such sequences are present. Yet another advantage of certain embodiments of the methods discussed herein is that they allow assessing the capacity of oocytes and sperm to undergo various processes affecting fertility, such as, but not limited to, meiosis, fertilization and cell differentiation. Thus, certain embodiments of the discussed methods allow identification of the specific biological processes that are diminished or defective in animals with reduced fertility. One more advantage of certain embodiments of the present methods is that they allow for assessment or evaluation of fertility of animals that have not yet reached sexual maturity or an age or stage when an animal can reproduce. Accordingly, animals can be selected for reproduction or stopped from reproducing based on their fertility characteristics.

### Diagnostic Methods

Diagnostic methods used in the embodiments of the method described herein include, but are not limited to, the following techniques: competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunocytochemistry, immunohistochemistry, PCR, quantitative PCR, real-time PCR, quantitative real-time PCR, in situ PCR of tissue or cell samples, and the like. The skilled artisan will understand that any antibody-based, nucleic acid-based, mass spectroscopy-based, FRET-based, or similar technique for detecting PGRMC1 levels can be used in the embodiments of the methods described herein.

It is appreciated, as exemplified by certain findings discussed herein, that PGRMC1 plays a role in one or more of oocyte and sperm production and function, gamete function, or embryonic development. In one embodiment of the present method, PGRMC1 testing is conducted in animals in order to assess fertility or in connection with assisted reproductive techniques.

### Fertility

Unless otherwise qualified, the term “fertility” refers generally to the natural capability of giving life. In live organisms, fertility is influenced by multiple biological processes, including, without limitation, gamete production, fertilization, embryonic development, or an ability to carry a pregnancy to term. Fertility is also influenced by various other factors, such as, but not limited to, nutrition, sexual behavior, culture, instinct, endocrinology, timing, living conditions or emotions. In animals, reproductive hormones participate in fertility regulation by various mechanisms. PGRMC1 is involved in such mechanisms. Mammals have hormonal cycles which determine when a female can achieve pregnancy or when a male is most virile.

Unless otherwise qualified, the term fertility encompasses definitions and uses of this term in medical, veterinary and biological areas. It is appreciated that the term fecundity can also be used to refer to fertility, for example, in the area or demographics, where “fecundity” is commonly defined as the potential for reproduction. Fecundity is included within the scope of the term “fertility,” as used herein. Fertility can be reduced or impaired by various factors generally discussed above as well as by other factors or processes.

It is understood that observable reproductive outcomes and/or reproductive success are included into the definition of fertility, as used herein. It is also understood that various metrics can be used to measure fertility. For example, one metric for monitoring reproductive success of cattle on a dairy farms is the so-called 21-day PR. In one example known in the field of dairy farming, the calculation of 21-day PR begins at the voluntary waiting period after calving (typically 45 to 60 days on most dairies). As each cow reaches the herd voluntary waiting period, she is eligible to become pregnant over the next 21-day interval. All eligible cows are monitored for pregnancy at 21-day intervals. If a cow is bred during a 21-day interval, she is no longer eligible until the next 21-day interval or she becomes pregnant to that breeding. The proportion of the cows becoming pregnant during each 21-day interval generates this herd reproductive measure. However, variations on the 21-day PR calculation process are also known in the field of dairy farming.

Unless otherwise qualified, infertility refers to deficient, lowered, reduced or impaired fertility, as well as to improbability to conceive. The term “infertility” can refer to the biologically reduced ability of an animal to contribute to conception, including reduced capacity for production of viable and functioning gametes, zygotes or embryos, as well as to the reduced capacity to carry pregnancy to full term. “Infertility” encompasses various definitions of infertility as used in the medical and veterinary areas. Terms such as, but not limited to, “subfertility,” “reduced fertility” or “impaired fertility,” are also included within the scope of the term “infertility.” Infertility includes both primary and secondary infertility. For example, the animals or breeding pairs that have never been able to conceive can be referred to as “having primary infertility,” while the term “secondary infertility” is often used to refer to difficulty conceiving after already having conceived.

The term “fertility treatment” is used to denote all methods that involve manipulation of fertility to achieve a desired reproductive result. An animal subjected to a fertility treatment can have normal, increased or reduced fertility. “Fertility treatment” as used herein can be used to manipulate fertility to achieve, for example, a desired genetic or other outcome, such as gender, a desired trait, or timing of reproduction, or reduction in a risk of infection. “Fertility treatment” encompasses “assisted reproductive technology” or “assisted reproductive technique,” which is used as a general term referring to methods used to achieve fertilization and/or pregnancy by artificial or partially artificial means. Artificial insemination (AI) is one example of an assisted reproductive technology or technique, and is a process by which sperm is placed into the reproductive tract of a female for the purpose of impregnating the female by using means other than sexual intercourse. In some instances of AI, freshly ejaculated sperm, or sperm which has been frozen and thawed, is placed in the cervix of a female, in a process known as intracervical insemination—ICI. In other instances, the sperm is placed a female's uterus, in a process known as intrauterine insemination—IUI. Another example of an assisted reproductive technology or technique is in vitro fertilization (IVF), which a term generally used to refer to a process by which egg cells are fertilized by sperm outside the womb, in vitro.

### Animals

Embodiments of methods of assessing or evaluating fertility an animal according to procedures, processes and schemes discussed herein are applicable to a variety of animals that express PGRMC1 or a similar protein. The term “animals,” as used herein, refers to Kingdom Animalia. Some embodiments of the methods disclosed herein are used to assess or evaluate fertility of vertebrate animals, meaning members of the subphylum Vertebrata, including, but not limited, to fishes, birds and tetrapods. Some other embodiments of the methods disclosed herein are used to assess or evaluate fertility of mammals, including, but not limited to the taxa of monotremes, marsupials and placentals. Placentals, or placental animals encompass without limitation, sloths, hyraxes, aardvarks, tenrecs, armadillos, anteaters, tree shrews, flying lemurs or colugos, lemurs, bushbabies, monkeys, apes, pikas, rabbits, hares, rodents, hedgehogs, gymnures, moles, shrews, solenodons, whales, dolphins, porpoises, even-toed ungulates, (including pigs, hippopotamus, camels, giraffe, deer, antelope, cattle, sheep and goats), bats, odd-toed ungulates (including horses, donkeys, zebras, tapirs, and rhinoceroses), pangolins or scaly anteaters and carnivorans.

In some embodiments, analysis of PGRMC1 and/or its variability is used to assess fertility of domesticated animals, including pets, livestock and farm animals. The term “livestock” includes cattle and refers to one or more domesticated animals raised and/or handled by humans in an agricultural setting to produce products, including food products, or labor. The term “livestock” includes poultry and fish. In other embodiments, analysis of PGRMC1 and/or its variability is used to assess fertility of undomesticated animals, including captive and wild animals. Undomesticated animals include, but are not limited too, pet animals, research animals, circus animals, amusement park animals, or any non-human animals inhabiting or placed in environments restricted or semi-restricted by humans, including, but not limited to, preserves, zoos, aquariums, amusement parks, safari parks or similar facilities. Assessing fertility of research animals is also contemplated according to the methods provided herein. Research animals include any animals used in animal experimentation, animal research, or animal testing, including conventional research animals, such as zebrafish, mice, rats, birds, fish, frogs and non-human primates.

Some embodiments of methods described herein usefully incorporate analysis of PGRMC1 and/or its variability into methods of breeding animals. The methods of breeding animals, or animal breeding, include methods of evaluating of the genetic value of animals, planned breeding for the purpose of producing animals possessing desired traits or lacking undesired traits, and methods of breeding animals in human controlled environments, with restricted settings, such as wildlife preserves, zoos and other conservation facilities (a term “captive breeding can also be used). In some other embodiments, analysis of PGRMC1 and/or its variability is usefully incorporated into animal research and management activities, including zoological research and animal conservation programs.

### Exemplar Embodiments

Some exemplary embodiments of methods disclosed herein are discussed below. In some methods, analysis of PGRMC1 and/or its variability is used to identify desirable or undesirable reproductive trait, feature or characteristic of an animal. An example of such a trait is fertility of an animal. Analysis of PGRMC1 and/or its variability is used to select animals with increased for further breeding in order to increase probability of positive reproductive outcomes in an individual animal or in a heard. One embodiment is a method of evaluating fertility of an animal, comprising obtaining a sample from the animal, determining a PGRMC1 characteristic in the sample, and comparing the determined PGRMC1 characteristic to a baseline PGRMC1 characteristic, wherein a variation between the determined PGRMC1 characteristic and the baseline characteristic indicates a level of fertility of the animal.

One more exemplary embodiment is a method of evaluating capacity of the animal to produce functional gametes, comprising determining a characteristic of PGRMC1 of the animal, wherein the characteristic indicates the capacity of the animal to produce the functional gametes. One more embodiment is a method of evaluating a probability of a successful reproductive outcome in an animal, including an animal that has not yet reached sexual maturity, comprising determining a characteristic of PGRMC1 of the animal, wherein the characteristic indicates the a capacity of the animal to achieve a successful reproductive outcome. One more embodiment is a method of evaluating an outcome of a fertility treatment in an animal patient comprising determining a characteristic of PGRMC1 of the animal, wherein the characteristic indicates the a capacity of the animal to produce functional gametes. Embodiments of the disclosed methods encompass variations where an animal is a male or a female, and the gametes are eggs or sperm.

One more exemplary embodiment is a method of improving fertility of a group of animals, comprising selecting an animal that has not reached sexual maturity from the group, obtaining a sample from the animal selected from the group, determining a PGRMC1 characteristic in the sample, comparing the determined PGRMC1 characteristic to a baseline PGRMC1 characteristic, wherein a variation between the determined PGRMC1 characteristic and the baseline characteristic indicates a level of fertility of the selected animal, and preventing the selected animal from further reproduction in an event the indicated level of fertility is lower than desired. In one variation of the above embodiment, the group of animals is a heard of cattle. In another variation of the above embodiment, the group of animal is a heard of dairy cattle. In one more variation of the method, the group of animals is a heard of dairy cows. In yet another variation, the selected animal is female. The selected animal, male or female, can be culled prior to reaching sexual maturity in order to prevent the selected animal from further reproduction in the event the indicated level of fertility is lower than desired

According to embodiments of the present methods, a PGRMC1 characteristic includes, but is not limited to one or more of the following: PGRMC1 expression, transcription, translation, amino acid sequence, nucleic acid sequence, post-translational modification, cell localization or tissue localization. In one example, PGRMC1 characteristic is a level of PGRMC1 expression, and the lowered PGRMC1 expression in an animal as compared to a reference population indicates reduced fertility of the animal as compared to the reference population. In another embodiment, the PGRMC1 characteristic to be analyzed or tested is a nucleic acid or a protein sequence and a variation of the nucleic acid or the protein sequence indicates reduced fertility of the animal.

### EXAMPLES

Embodiments of the present methods are further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention.

Experimental procedures described in this and other examples are known to those of ordinary skill in the art and are described, for example, in the articles in Peluso I, Peluso et al. “Regulation of ovarian cancer cell viability and sensitivity to cisplatin by progesterone receptor membrane component-1,” J. Clin. Endocrinol. Metab. 93:1592-1599, 2008 (“Peluso II”); Peluso et al. “Progesterone activates a progesterone receptor membrane component 1-dependent mechanism that promotes human granulosa/luteal cell survival but not progesterone secretion,” J. Clin. Endocrinol. Metab., 94:2644-2649, 2009 (“Peluso III”); Peluso et al. “Progesterone Membrane Receptor Component I Expression in the Immature Rat Ovary and Its Role in Mediating Progesterone's Antiapoptotic Action,” Endocrinology, 147:3133-3140 (“Peluso IV”), as well as in other sources referenced below.

### Example 1

### Localization of PGRMC1 in Bovine and Mouse Oocytes

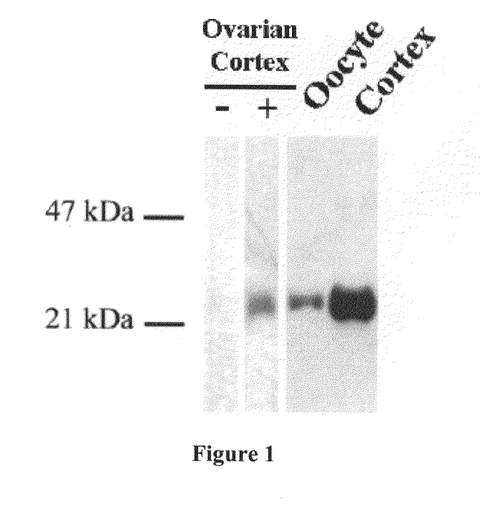

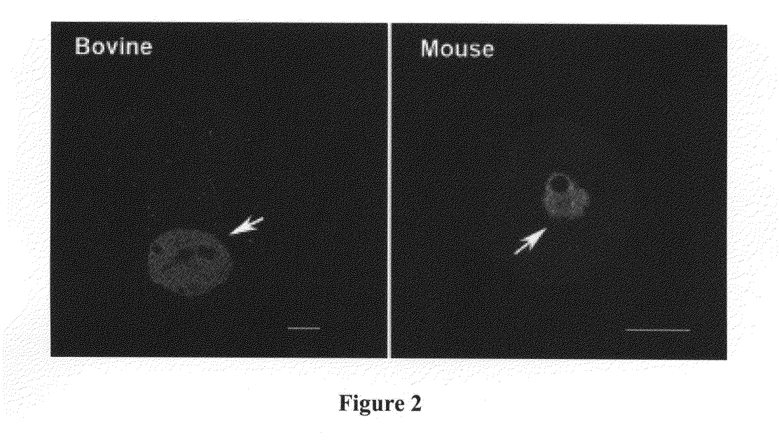

A Western blot analysis with an anti-PGRMC1 antibody (Prestige Antibodies Cat. No. HPA002877, Sigma Chemical Co. St. Louis, Mo.) demonstrated that PGRMC1 was specifically detected as an approximately 26 kDa band in bovine oocytes, as shown in FIG. 1, thus establishing that the antibody specifically detected bovine PGRMC1. The antibody was then used in antibody cell-imaging studies to localize PGRMC1 in oocytes, zygotes and blastocysts. In particular, cell imaging determined localization of PGRMC1 in germinal vesicle stage bovine and mouse oocytes. As seen in FIG. 2, PGRMC1 (shown in red) was highly concentrated within the germinal vesicle of both bovine and mouse oocytes. Similar studies were conducted with other PGRMC1 antibodies using other types of cells and provided similar findings.

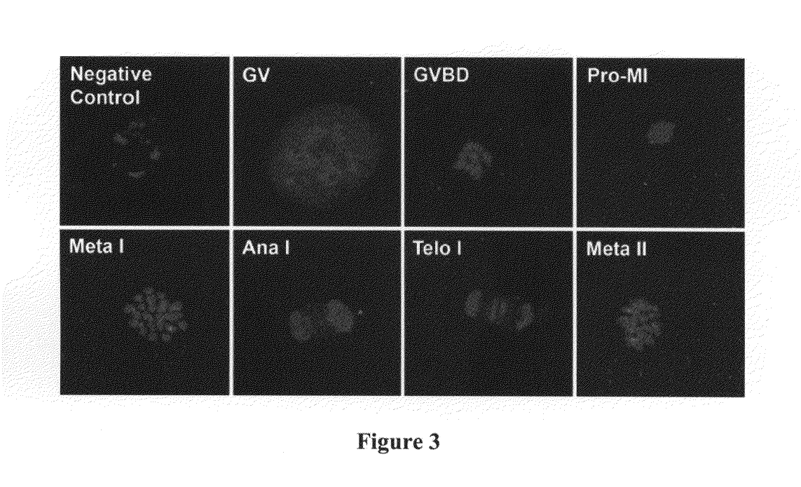

Cell-imaging analysis of localization of PGRMC1 during bovine oocyte maturation, as shown in FIG. 3, revealed a relationship between PGRMC1 (shown in red) and chromatin (shown in blue). Bovine oocytes were collected at the germinal vesicle stage, after the breakdown of the GV, prometaphase 1, metaphase I, anaphase I, telophase I and metaphase II. In the germinal vesicle stage, PGRMC1 does not interact with the chromatin. At prometaphase I, PGRMC1 started to interact with the chromatin. At metaphase I, it was detected throughout each chromosome, as indicated by the pink-purple staining. After the chromosomes separate in anaphase I and telophase I, PGRMC1 dissociated from the chromosomes and concentrated between them. Finally, in metaphase II, PGRMC1 re-associated with the chromosomes at focal points near the apparent centromeric region of each chromosome. These sequential changes in the localization of PGRMC1 indicated that PGRMC1 plays a role in chromosome separation. The localization of PGRMC1 to focal points near the apparent centromeric region of each chromosome indicated that PGRMC1 colocalizes with the centromere.

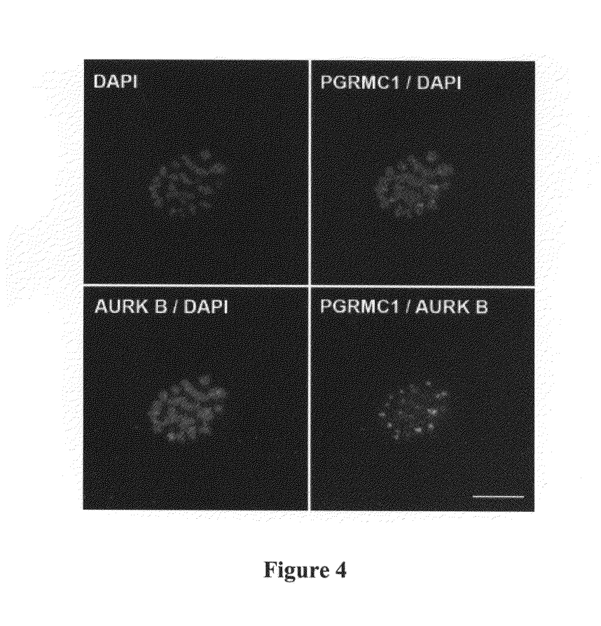

Cell-imaging studies of co-localization of PGRMC1 with Aurora B, a kinase and a well-characterized component of the chromosomal passenger complex that associates with the centromeres, demonstrated that PGRMC1 localizes to Aurora B, as shown in FIG. 4. A centromere is known to be the site at which the kinetochore forms to allow the attachment of spindle fibers for the separation of the chromosomes. The experimental results discussed in this example indicated that that PGRMC1 plays an important role in regulating oocyte maturation.

### Example 2

### Localization of PGRMC1 in a Bovine Zygote

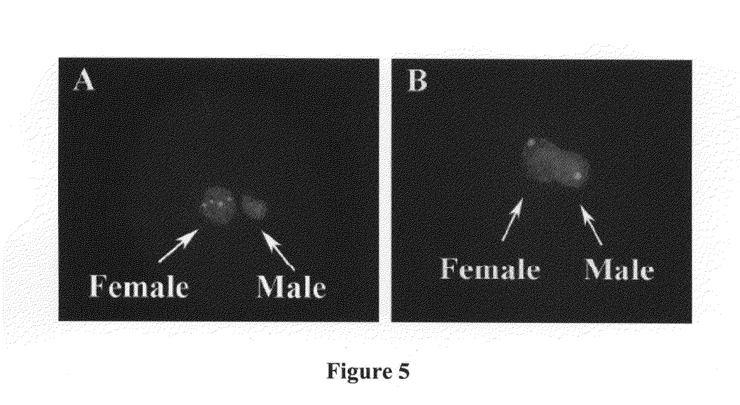

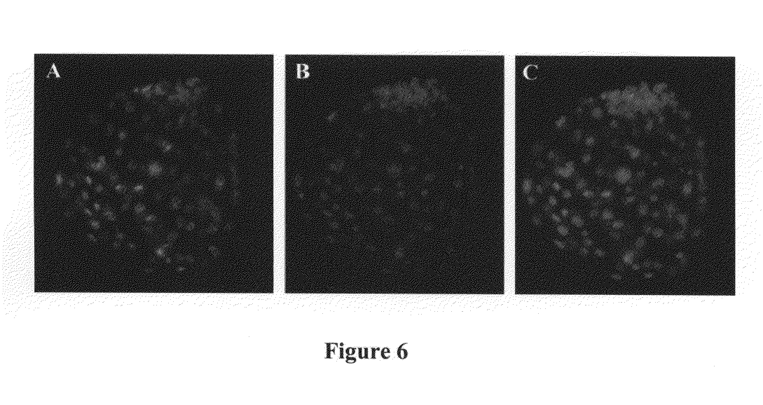

Cell-imaging studies images of PGRMC1 localization in a bovine zygote were performed. As shown in FIG. 5, after in vitro fertilization, PGRMC1 localized almost exclusively to nucleolar-like structures within the female and male pronuclei. As shown in FIG. 6, in blastocysts, PGRMC1 is expressed in virtually all of the cells. The stage-dependent changes in PGRMC1 expression and localization described in this example indicated that PGRMC1 plays important roles in fertilization and early embryonic development as well as in oocyte maturation.

### Example 3

### Role of PGRMC1 in Oocyte Maturation

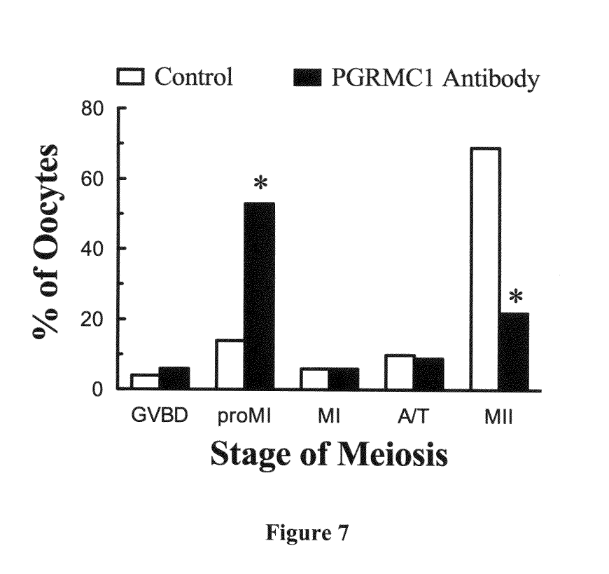

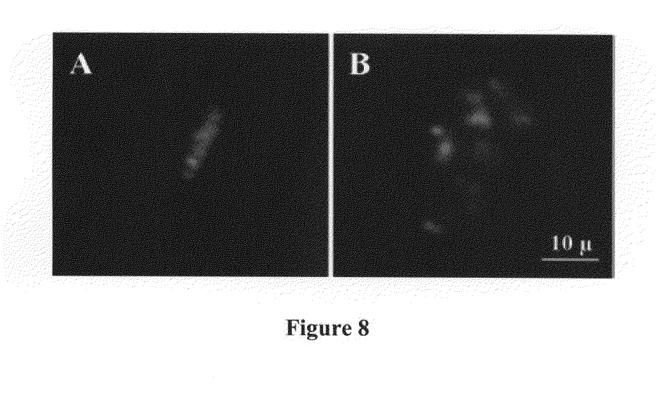

Germinal vesicle (GV) stage bovine oocytes within the cumulus cell mass were injected with an antibody to PGRMC1(0.3 μM; Sigma Chemical Co, St. Louis, Mo.). Control cells were injected with 0.3 μM IgG. The oocyte-cumulus cell complexes were incubated for 24 hours, then the cumulus cells were removed and the oocytes were assessed for the stage of meiosis. As shown in FIG. 7, 70% of the control oocytes matured to the metaphase II stage. However, only 22% of the PGRMC1 antibody-injected oocytes reached the metaphase II stage (p<0.05). Most of these oocytes were arrested in prometaphase I stage. Some of the PGRMC1 antibody-injected oocytes progressed to metaphase I or II, but cell-imaging studies showed that the metaphase plates of these cells were disorganized, and the chromosomes appeared scattered, as shown in FIG. 8. The experimental results described in this example confirm that PGRMC1 plays a role in oocyte maturation.

### Example 4

### PGRMC1-GFP Expression Vector

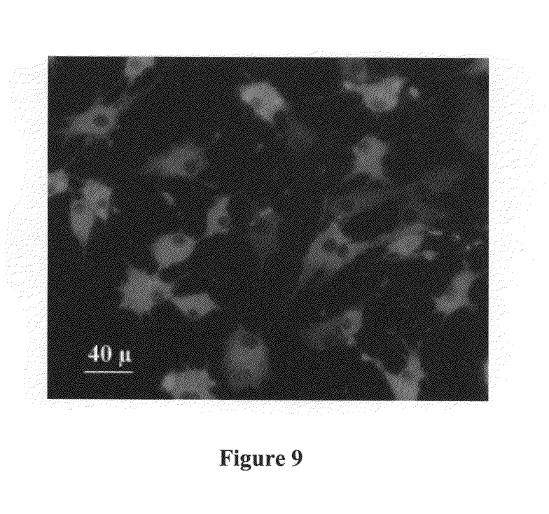

An adenovirus-PGRMC1-GFP expression vector was prepared by isolating total RNA from GL5 cells, a human granulosa cell line, to generate cDNA. The PGRMC1 open reading frame was amplified by PCR. The primers, described in Mansouri et al., “Alterations in the expression, structure and function of progesterone reeptor membrane component-1 (PGRMC1) in premature ovarian failure,” Hum. Mol. Gen. 17:3776-3783, 2008, contained an XhoI and a HindIII sites at the ends to facilitate, cloning into the pShuttle-CMV vector. Co-transfection of the linearized pShuttle-CMV-PGRMC1 and pAdTrack DNA was then performed. Viral stocks were amplified, titered and stored at −80° C. As shown in FIG. 9, infection with this adenoviral construct at a MOI of 1×10⁻⁷ was very effective, resulting in nearly 100% of the human granulosa/luteal cells expressing PGRMC1-GFP fusion. Treatment with the adenoviral-PGRMC1-GFP expression vector increased protein levels of PGRMC1-GFP by several fold compared to endogenous PGRMC1 levels. Once transfected, the PGRMC1-GFP continued to be expressed for at least 72 hours post-infection.

### Example 5

### Isolation of Bovine PGRMC1 mRNA

A bovine blood sample was collected and lymphocytes isolated by centrifuging the sample through a Ficoll gradient. RNA was then isolated and assayed for the presence of PGRMC1 using the following primer pairs:

(SEQ ID NO: 3)

| | |
|---|---|
| Forward | 5′-GCC TGG ATA AGG AAG CAC TG-3′ |

(SEQ ID NO: 4)

| | |
|---|---|
| Reverse | 5′- ATC ATT CTT CCG AGT GCT CTC -3′ |

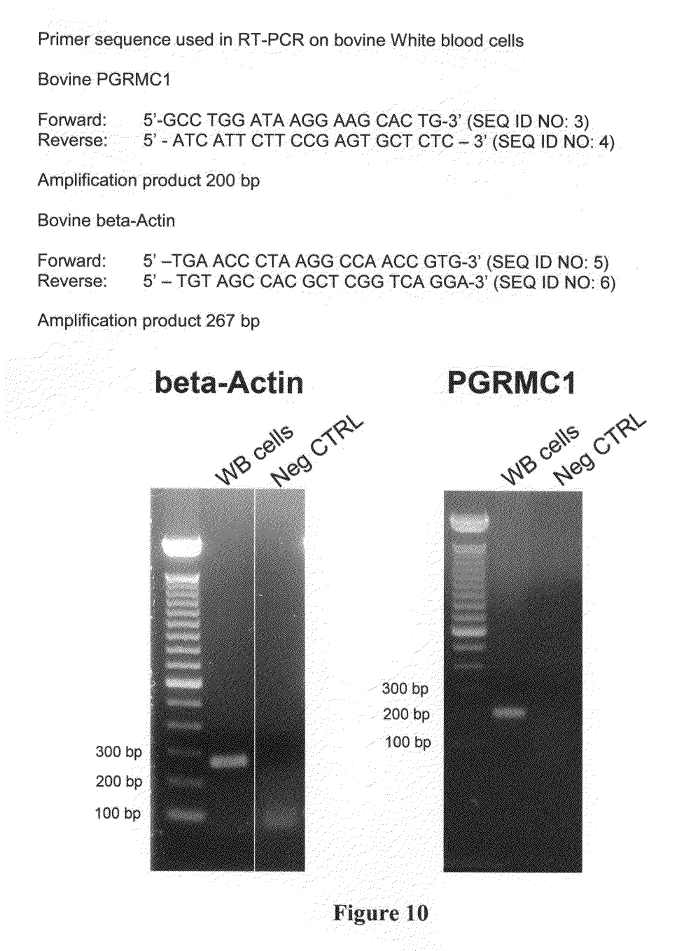

As shown in FIG. 10, a PCR product of the predicted size of 200 bp was detected. The product was extracted from the gel and subjected to sequence analysis, which confirmed that this product was bovine PGRMC1. The actin PCR product is shown as a control.

### Example 6

### A Method to Determine Functional Significance of PGRMC1 Mutations

Physiological importance of PGRMC1 mutations, including, but not limited to the mutations corresponding to known substitutions H165R and D120G in human PGRMC1, is assessed by making PGRMC1 fusion proteins (“fusion proteins”) and injecting them into germinal vesicle stage oocytes or metaphase II oocytes. Alternatively these fusion proteins are transfected into any suitable target cell. In one example, fusion proteins are fusions of wild-type or mutant PGRMC1 sequences with a green fluorescent protein (GFP). If GFP fusion proteins are tested, then GFP is injected as a negative control. It is understood that negative controls are selected based on the type of a fusion protein. Cell-imaging studies according to procedures known to those of ordinary skill in the art are used to monitor localization of the wild-type and mutant fusion proteins and the ability of the injected oocytes to undergo in vitro maturation, fertilization and embryonic development, as discussed in more detail below.

Expression vectors for the wild type and mutant fusion proteins (“expression vectors”) were or are prepared using conventional molecular biology techniques, for example, as described in Peluso I, and elsewhere in the present document. The fusion proteins and the negative control, at a concentration of approximately 10 pg in 10 pl, are injected into germinal vesicle stage oocytes as describe in Gordo et al., “Injection of sperm cytosolic factor into mouse metaphase II oocytes induces different developmental fates according to the frequency of [Ca(2+)](i) oscillations and oocyte age,” Biol. Reprod., 62:1370-1379, 2000. The injections result in final intracellular concentrations of the injected material of 0.3 pg/oocyte. After the injections, the oocytes are cultured for 24 hours. The Hoechst 33342 dye is used to stain DNA in living oocytes according to known procedures, described, for example, in Cao, thereby maximizing the GFP fluorescence and still allowing for the determination of the stage of maturation. To determine if the PGRMC1 fusion protein interacts with the DNA, the living oocytes are observed under confocal optics and images are obtained of the fusion protein merged with the DNA.

In order to determine appropriate experimental conditions, injections of various dosages of the wild-type fusion protein are tested and the effectiveness is observed Effectiveness of the injection is determined by monitoring two endpoints. First, the effect of the purified PGRMC1 fusion protein on oocyte maturation, fertilization and embryonic development is monitored. Second, the ability of the wild-type fusion protein to localize to the kinetochore/centromere complex is assessed. If wild-type fusion protein localizes to the kinetochore/centromere complex, thereby mimicking endogenous PGRMC1, then the injection is considered functional. Thus identified experimental conditions are used to assess the effect of the mutated PGRMC1 fusion protein.

If injections of wild-type PGRMC1 fusion protein do not appear to mimic endogenous PGRMC1, troubleshooting of potential experimental problems is performed. For example, in order to overcome a potential problem that the function of the injected fusion protein is adversely affected because it is not endogenously synthesized within the oocytes, injection of expression vectors into oocytes can be employed. For this application, the expression vectors are modified by extending the polyA tail to facilitate protein synthesis in germinal-vesicle stage oocytes, as described, for example, in Aida et al., “Expression of a green fluorescent protein variant in mouse oocytes by injection of RNA with an added long poly(A) tail,” Mol. Hum. Reprod., 7(11):1039-1046, 2001. Oocyte maturation can be delayed, as needed, by the addition of cAMP analogs, in order to ensure sufficient time to achieve adequate levels of protein expression.

In another example of a potential experimental problem, wild-type PGRMC1 fusion protein fails to localize to the chromosomes, as has been shown for endogenous PGRMC1, due to the presence of the GFP-tag at the C-terminus. It is recognized, for example, that GFP's presence at the C-terminus might interfere with PGRMC1's interaction with chromosomes. PGRMC1 expression vectors and fusion proteins are therefore generated, in which the GFP-tag is placed on the N-terminus. It is also recognized that a relatively large size (approximately 26 kDa) of the GFP tag can influence its ability of the fusion proteins to localize to the chromosomes. A smaller tag, such as the amino acid sequence YPYDVPDYA, which is known as an HA, can therefore be used to tag the exogenous PGRMC1, and the oocytes are fixed and co-stained with an antibody to HA and DAPI.

Another example of a potential experimental problem to be addressed is depletion of endogenous PGRMC1 levels in order to observe the effects of the experimentally introduced fusion proteins. The depletion is achieved by PGRMC1 siRNA treatment, based on the experimental approaches discussed, for example, in Peluso I, Peluso II and III. An alternative to siRNA treatment is the development of a transgenic mouse in which PGRMC1 is depleted conditionally from the oocyte.

Experimental studies according to the procedures similar to those described in the present document are conducted. Cell-imaging studies monitoring changes are conducted to evaluate colocalization of mutant PGRMC1 at certain stages of oocyte maturation, fertilization and early preimplantation development with one or more cell structures known to be functionally significant in this process in order to assess functional significance of PGRMC1 mutations.

### Example 7

### Correlative Analysis of PGRMC1 Expression and Structure in Cows of Reduced Fertility

Bovine ovaries are obtained from aged-matched cows at the time of slaughter. Two types of ovaries are identified based on gross morphology: “normal” ovaries and those showing signs of premature ovarian failure (i.e. small and with few follicles). Ovaries from cows with premature ovarian failure appear small and have relatively few follicles (for examples, less than ten follicles per ovary) greater than 6 mm in diameter, as compared to “normal” ovaries. Detailed histological analysis of the ovaries from cows with premature ovarian failure also reveals that they contain, on average, fewer primary follicles than “normal” ovaries, and contain more stroma and connective tissue. One hundred ovaries with normal morphological features, designated as “control” ovaries, and one hundred ovaries with premature ovarian failure features are analyzed as outlined in the following manner.

One ovary from each animal is used to confirm classification based on histological analysis. DNA, RNA and protein are isolated from the remaining ovary. DNA, RNA and protein isolated using the TrioMol isolation kit from GenScript (Piscataway, N.J.). This kit can be used to isolate DNA, RNA and protein from the same ovarian sample and the isolated DNA/RNA and protein are suitable for PCR and Western blot protocols, respectively. DNA, RNA and protein isolated from the ovaries are analyzed as outlined below.

Analyses of PGRMC1 mRNA, genetic structure and protein levels are conducted. PGRMC1 is assessed by at least three different procedures. First, some of the isolated RNA from each sample is used to determine the amount of mRNA by real time PCR using a known protocol by real time PCR using a protocol similar to a published protocol, described, for example, in Peluso III. PGRMC1 mRNA levels are normalized against actin as a control. Thus obtained experimental data on the mRNA amounts provides information on the amount of expressed PGRMC1. DNA is used in a PCR protocol that amplifies the entire coding sequence of PGRMC1 (i.e. Exons 1-3). The PCR products are sequenced, which provides information on the genetic structure of PGRMC1.

The protein isolated from each sample is processed for Western blot analysis, using known protocols. PGRMC1 antibody, such as a rabbit antibody commercially available from Sigma Chemical Co., and anti-rabbit IR Dye 800 (Li-Cor Bioscience, Lincoln, Nebr.) are used as the primary and secondary antibodies, respectively. The Western blot is simultaneously probed with a GAPDH mouse primary antibody and an anti-mouse IRDye 700 secondary antibody. The Western blot is imaged in a quantitative manner using the Odyssey Infrared imaging system from Li-Cor Bioscience (Lincoln, Nebr.). GAPDH serves as a loading control. This analysis provides information on an amount of PGRMC1 expressed and on the changes in its molecular weight. If changes in the molecular weight are observed, these post-translational modifications such as phosphorylations, are assessed according to known experimental procedures. Characteristics of PGRMC1 expression and structure are identified that are associated with premature ovarian failure and reduced fertility in cows.

### Example 8

### Determination of Predictive Value of PGRMC1 Variation or Reduced Fertility

Blood samples are taken from 300 cows two years of age or younger. Lymphocytes are isolated and assessed for alterations in PGRMC1 characteristics, including DNA, RNA and protein levels, according to known procedures including those discussed elsewhere herein. In addition, the fertility records are assessed once the cow is two years of age and older. Characteristics of PGRMC1 gene structure are identified that are predictive of premature ovarian failure and reduced fertility in cows.

Analysis of PGRMC1 mRNA, genetic structure and protein levels are conducted. PGRMC1 is assessed by at least three different procedures. First, some of the isolated RNA from each sample is used to determine the amount of mRNA by real time PCR using a known protocol. PGRMC1 mRNA levels are normalized against actin as a control. Thus obtained experimental data on the mRNA amounts provides information on the amount of expressed PGRMC1. DNA is used in a PCR protocol amplifies the entire coding sequence of PGRMC1 (i.e. Exons 1-3). The PCR products are sequenced, which provides information on the genetic structure of PGRMC1.

The protein isolated from each sample is processed for Western blot analysis using known procedures. PGRMC1 antibody, such as a rabbit antibody commercially available from Sigma Chemical Co., and anti-rabbit IR Dye 800 (Li-Cor Bioscience, Lincoln, Nebr.) are used as the primary and secondary antibodies, respectively. The blot is simultaneously probed with a GAPDH mouse primary antibody and an anti-mouse IRDye 700 secondary antibody. The Western blots are imaged in a quantitative manner using the Odyssey Infrared imaging system from Li-Cor Bioscience (Lincoln, Nebr.). GAPDH serves as a loading control. This analysis provides information on an amount of PGRMC1 expressed and on the changes in its molecular weight. If changes in the molecular weight are observed, these post-translational modifications such as phosphorylations, are assessed according to known experimental procedures.

The experimental data are statistically analyzed. Functional significance of those genetic alterations of PGRMC1 that are not suitable for statistical analysis because they are not observed at a high enough frequency is assessed as outlined elsewhere in the present document.

The experimental results obtained from at least some of the studies described above indicate that certain genetic changes in PGRMC1 alter a bovine fertility, for example, a cow's ability to generate fertilizable oocytes. Mutations in PGRMC1 affecting fertility or an ability to generate fertilizable oocytes are identified.

### Example 9

### A Test to Assess Fertility in a Cow

A test is developed that determines, based on a sample obtained from a cow, whether the cow's PGRMC1 contains variations that correlate with the reduced fertility. The results obtained from such a test may be used to calculate the values evaluating predicted fertility in cows. A dairy farmer may use the predicted fertility to select cows with desired fertility characteristics for a dairy herd.

While this invention has been described in detail with regard to embodiments thereof, it should be understood that variations and modifications can be made without departing from the spirit and scope of the invention described herein and/or defined in the following claims.
All the documents cited herein are incorporated by reference in their entirety.

What is claimed is:

1. A method of evaluating a level of fertility of an animal comprising:
obtaining a sample from the animal;
determining a PGRMC1 characteristic in the sample; and,
comparing the determined PGRMC1 characteristic to a baseline PGRMC1 characteristic,
wherein a variation between the determined PGRMC1 characteristic and the baseline characteristic indicates the level of fertility of the animal.

2. The method of claim 1, wherein the PGRMC1 characteristic is one or more of PGRMC1 expression, transcription, translation, amino acid sequence, nucleic acid sequence, post-translational modification, cell localization or tissue localization.

3. The method of claim 1, wherein the sample is a cell sample or a tissue sample.

4. The method of claim 1, wherein the sample is a blood sample, a lymphocyte sample, an oocyte sample, or a sperm sample.

5. The method of claim 1, wherein the animal is a female.

6. The method of claim 1, wherein the animal is a male.

7. The method of claim 1, wherein the PGRMC1 characteristic is a level of PGRMC1 expression and wherein the variation is a lowered expression that indicates reduced fertility of the animal.

8. The method of claim 1, wherein the PGRMC1 characteristic is a nucleic acid sequence and the variation is a variation of the nucleic acid sequence that indicates reduced fertility of the animal.

9. The method of claim 1, wherein fertility is a capacity of the animal to produce functional gametes.

10. The method of claim 1, wherein the animal is a cow.

11. A method of evaluating capacity of an animal to produce functional gametes, comprising determining a characteristic of PGRMC1 of the animal, wherein the characteristic indicates the capacity of the animal to produce the functional gametes.

12. The method of claim 11, wherein the animal is a female and the functional gametes are fertilizable oocytes.

13. The method of claim 11, wherein the PGRMC1 characteristic is one or more of PGRMC1 expression, transcription, translation, amino acid sequence, nucleic acid sequence, post-translational modification, cell localization or tissue localization.

14. A method of improving fertility of a group of animals, comprising:
selecting an animal that has not reached sexual maturity from the group;
obtaining a sample from the animal selected from the group;
determining a PGRMC1 characteristic in the sample;
comparing the determined PGRMC1 characteristic to a baseline PGRMC1 characteristic, wherein a variation between the determined PGRMC1 characteristic and the baseline characteristic indicates a level of fertility of the selected animal; and,
preventing the selected animal from further reproduction in the event that the indicated level of fertility in the selected animal is lower than desired.

15. The method of claim 14, wherein the group of animals is a herd of cattle.

16. The method of claim 15, wherein the group of animals is a herd of dairy cattle.

17. The method of claim 14, wherein the group of animals is a herd of dairy cows.

18. The method of claim 14, wherein the selected animal is a female.

19. The method of claim 14, wherein the preventing the selected animal from further reproduction comprises culling the selected animal from the group of animals prior to the selected animal's reaching sexual maturity.

20. The method of claim 14, wherein the PGRMC1 characteristic is one or more of PGRMC1 expression, transcription, translation, amino acid sequence, nucleic acid sequence, post-translational modification, cell localization or tissue localization.


SEQUENCE LISTING (6 sequences)

SEQ ID NO: 1 - protein, 194 aa, Bos taurus
MAAEDVAATGADTSELESGGLLQEIFTSPLNLLLLGLCIFLLYKIVRGDQPAASDSDDDEPPPLPRLKRRDFTPAELRRFDGVQDPRILMAINGKVFDVTKGRKFYGPEGPYGVFAGRDASRGLATFCLDKEALKDEYDDLSDLTPAQQETLSDWDSQFTFKYHHVGKLLKDGEEPTVYSDEEEPKDESTRKND

SEQ ID NO: 2 - DNA, 582 bp, Bos taurus
atggctgccgaggatgtggcggcgaccggcgcagacacgagcgagctcgagagcggcgggctgctgcaagagattttcacgtcgccgctcaacctgctgctccttggcctttgcatctttctgctctacaagatcgtgcgcggggaccagccggcggccagcgatagcgacgacgacgagccgcccccgctgccccgccttaagcggcgcgacttcacccctgccgagctgcggcgcttcgacggcgtacaggacccgcgtatactcatggccatcaatggcaaggtgttcgacgtaaccaaaggccgcaagttctacgggcctgaggggccgtatggagtctttgctggaagagatgcatccagaggccttgccaccttttgcctggataaggaagcgctgaaggatgagtacgatgacctttccgacctcactcctgcccagcaggagaccctgagtgactgggactctcagttcactttcaagtaccatcatgtgggcaaactgctgaaggacggggaggagcccaccgtgtactcagacgaggaggagccaaaagatgagagcactcggaagaatgat

SEQ ID NO: 3 - DNA, 20 bp, Artificial Sequence
  Primer
gcctggataaggaagcactg

SEQ ID NO: 4 - DNA, 21 bp, Artificial Sequence
  Primer
atcattcttccgagtgctctc

SEQ ID NO: 5 - DNA, 21 bp, Artificial Sequence
  Primer
tgaaccctaaggccaaccgtg

SEQ ID NO: 6 - DNA, 21 bp, Artificial Sequence
  Primer
tgtagccacgctcggtcagga